(19) United States
(12) Patent Application Publication
(10) Pub. No.: US 2003/0049223 A1
(43) Pub. Date: Mar. 13, 2003

(54) Hair care compositions including arabinogalactan and methods of use

(76) Inventors: Kristen Kerns, New Brighton, MN (US); Morton A. Westman, Oak Brook, IL (US)

Correspondence Address: MUETING, RAASCH & GEBHARDT, P.A., P.O. BOX 581415, MINNEAPOLIS, MN, 55458, US

(21) Appl. No.: 09/804,133

(22) Filed: Mar. 12, 2001

(51) Int. Cl. A61K 7/06; A61K 7/11

(52) U.S. Cl. 424/70.13

(57) ABSTRACT

The present invention provides compositions including an arabinogalactan and a hair holding polymer, where the arabinogalactan and the hair holding polymer are present in an amount effective to increase curl retention. The present invention also provides methods of using the compositions, including methods for increasing curl retention and methods for styling hair.

### BACKGROUND

Hair care products are commonly employed to enhance the ability of hair to retain a desired configuration or coiffure. Such hair care products include styling aids which are used in the process of styling hair into a desired configuration. Styling aids include, for instance, setting lotions, setting gels, and mousses. Hair care products also include those that can be used for hair holding. The primary functional materials used in these types of hair styling products are generally resins, gums, and polymers which are capable of imparting style or shape to the hair and/or retaining it in that style or shape. A high level of curl retention, or hold, is generally desirable, particularly under humid conditions. Unfortunately, many hair styling products having good hold characteristics suffer from the disadvantage of losing curl retention or hold under humid conditions.

### SUMMARY OF THE INVENTION

There remains a need for hair care products that can provide increased curl retention. The present invention represents an advance in the art of hair care products that can protect hair from the effects of increased humidity, including loss of curl retention. When arabinogalactan and a hair holding polymer were combined and applied to hair, there was a surprising and unexpected result under high humidity conditions: the components of the composition acted synergistically to provide increased curl retention.

Accordingly, the present invention provides compositions that include an arabinogalactan and a hair holding polymer, and methods of using the compositions. The arabinogalactan and the hair holding polymer are present in an amount effective to increase curl retention. The arabinogalactan, which is preferably ultrarefined, is also preferably obtained from a tree of the genus Larix. The compositions preferably include at least about 1% by weight arabinogalactan based on the total weight of the hair care composition.

The hair holding polymer is preferably a polyvinylpyrrolidone-based polymer, an acidic-vinyl-ether-based polymer, a acidic-polyvinyl-acetate-based polymer, a acidic acrylic polymer, a basic acrylic polymer, a cellulose derivative, a chitin derivative, a chitosan derivative, an amphoteric acrylic polymer, or combinations thereof. An example of a polyvinylpyrrolidone-based polymer is vinylpyrrolidone/vinyl acetate copolymer. Preferably, the vinylpyrrolidone/vinyl acetate copolymer has a vinylpyrrolidone to vinyl acetate mole ratio of about 70 to about 30 moles vinylpyrrolidone to about 30 to about 70 moles vinyl acetate. The composition preferably include at least about 0.1% by weight hair holding polymer based on the total weight of the hair care composition.

The composition is preferably in the form of a hair care product, including, for instance, a setting lotion, a setting gel, a mousse, a hair spray, or a pommade. The composition preferably further includes a plasticizer.

The composition of the present invention is preferably used in a method for increasing curl retention. The method includes contacting hair with an amount of the composition effective to increase curl retention, and allowing the hair to dry. The method preferably also includes styling the hair before, during, or after contacting the hair with the composition.

The present invention also provides a method for styling hair. The method includes styling hair, contacting hair with an amount of the composition of the present invention effective to increase curl retention, and allowing the hair to dry. The hair is preferably styled before contacting the hair with the composition, while contacting the hair with the composition, or after contacting the hair with the composition.

Unless otherwise specified, “a,” “an,” “the,” and “at least one” are used interchangeably and mean one or more than one.

### DETAILED DESCRIPTION OF PREFERRED EMBODIMENTS OF THE INVENTION

The present invention provides compositions that include an arabinogalactan and a hair holding polymer. Preferably, the arabinogalactan and hair holding polymer are present in the composition in an amount effective to increase curl retention. Curl retention is a measurement of the holding power of a composition that is applied to hair, i.e., the ability of a composition to keep hair in a desired configuration or coiffure. As used herein, the terms “increased curl retention” and “increased percent curl retention” refer to the ability of a composition to keep hair in a desired configuration under defined conditions when compared to hair without the composition, or hair to which the individual components of the composition are applied. Compositions of the present invention result in a synergistic increase in curl retention under high humidity conditions. The increase is synergistic in that the level of curl retention is greater than the level of curl retention observed when only the arabinogalactan or the hair holding polymer is applied. It is expected that the compositions of the present invention also result in a synergistic increase under low humidity conditions. Without intending to be limited by theory, it is believed that the increase in curl retention is due to an increase in shielding hair and/or the holding polymer from moisture. Shielding hair from moisture means preventing H₂O from disrupting hydrogen bonds present in hair.

Curl retention is preferably measured by methods known to the art, and preferably by measuring percent curl retention as described in the Examples. Preferably the hair is human hair, and preferably the hair used in a test is from the same lot. Typically, the hair is washed and allowed to dry before being used. The length of the hair when extended is measured. This measurement is referred to as “L” in the equation below.

After the composition is applied to the hair, the excess is removed and the hair is wound on a mandrel of appropriate diameter. The test composition can be applied by immersion of the hair in the composition, by spraying (either aerosol or non-aerosol) the composition on the hair, or by manually applying the composition (for instance, as a gel). After the hair has dried, the mandrel is removed, and the length of the curled hair is measured (“Lo” in the equation below). The curled hair is exposed to humidity for various periods of time at a constant temperature, preferably about 23° C. Preferably, the hair is placed in an environmental chamber that allows the temperature and humidity to be controlled. Low humidity conditions are typically about 30% to about 45% relative humidity. High humidity conditions are typically about 70% to about 90% relative humidity, preferably, about 90% relative humidity. Relative humidity can be measured using a hygrometer or a humidity meter. After the appropriate period of time has elapsed, the hair is measured to determine the length to which the hair has extended or drooped (“Lt” in the equation below), and the percent curl retention for the hair is determined by using the following equation:

$$\frac{L - {L}_{t}}{L - {L}_{o}} \times 100 = \% curl retention ,$$

where L=Length of fully extended hair, Lₜ=Length of curl after exposure at time (t), and Lₒ=Length of curl before exposure. Preferably, the amount of time the hair is exposed to the low humidity or high humidity conditions is about 30 minutes to about 80 minutes, more preferably about 45 minutes.

Compositions of the present invention result in a percent curl retention that is greater than the percent curl retention that occurs when the arabinogalactan and the hair holding polymer are used separately. Accordingly, to determine if a composition results in an increased percent curl retention, the percent curl retention that occurs from using a composition including arabinogalactan and a hair holding polymer is determined and compared to the percent curl retention that results from using the arabinogalactan and the hair holding polymer alone. Preferably, high humidity conditions are used. Preferably, the increase in percent curl retention is at least about 1.2 times greater, most preferably at least about 1.5 times greater, than the highest percent curl retention observed with either the arabinogalactan or the hair holding polymer alone.

1. Arabinogalactan

The present invention provides compositions that include an arabinogalactan. Preferably, the arabinogalactan is not derivatized. Arabinogalactan is a water-soluble polysaccharide that is preferably isolated from species of the genus Larix. Arabinogalactan may constitute up to 35% of the total heartwood of some species (Stout, “Larch Arabinogalactan” in Industrial Gums, R. L. Whistler Ed., Academic Press, New York, pp. 307-310, 1959). Arabinogalactan is highly soluble and may be obtained at 95% purity from larch chips. In some aspects of the present invention, ultrarefined arabinogalactan (i.e., highly purified) is used. One method for the preparation of ultrarefined arabinogalactan is disclosed in U.S. Pat. No. 5,116,969 (Adams et al.). Ultrarefined arabinogalactan of greater than 95%, or optionally, greater than 99.9% purity is preferably used. A suitable example is commercially available under the trade designations LAREX UF and LARACARE A200, both from Larex, Inc., St. Paul, Minn. Ultrarefined arabinogalactan is highly stable, non-toxic, and highly water soluble. In another embodiment, arabinogalactan material is used as produced and described in U.S. Pat. No. 5,756,098 (Price et al.).

A variety of arabinogalactans may be used in this invention. In one embodiment, the molecular weight of the arabinogalactan ranges from about 6,000 Daltons to about 2,500,000 Daltons. In another embodiment, the molecular weight of the arabinogalactan used is about 6,000 Daltons to about 300,000 Daltons, and in another embodiment about 10,000 Daltons to about 30,000 Daltons, as assessed by size exclusion liquid chromatography using Pullulan reference standards available from Millipore Corporation (Milford, Mass.)

A composition of the invention preferably includes at least about 1% by weight arabinogalactan, more preferably at least about 2.5% by weight arabinogalactan, where “% by weight arabinogalactan” refers to the percent weight based on the total weight of the composition. Preferably, a composition of the invention includes no greater than about 5% by weight, more preferably no greater than about 10% by weight, arabinogalactan. Most preferably, a composition of the invention includes about 2.5% by weight of arabinogalactan.

2. Hair Holding Polymers

A composition of the invention also includes a hair holding polymer. A hair holding polymer is a polymer that may be used to enhance the ability of hair to retain a desired configuration or coiffure. A hair holding polymer may be natural (i.e., it is present in or produced by nature) or synthetic (i.e., it is artificial or man-made). A hair holding polymer may be a homopolymer, a copolymer, or a terpolymer. A copolymer is a polymer with two or more different monomer units. Various combinations of hair holding polymers may be used.

Examples of hair holding polymers include polyvinylpyrrolidone-based polymers such as polyvinylpyrrolidones, polyvinylpyrrolidone/vinyl acetate copolymers, polyinylpyrrolidone/vinyl acetate/vinyl propionate terpolymers, polyvinylpyrrolidone/alkyl aminoacrylate copolymers, polyvinylpyrrolidone/acrylate/(meth)acrylic acid copolymers, and polyvinylpyrrolidone/alkyl aminoacrylate/vinyl caprolactam terpolymers. Suitable examples of polyvinylpyrrolidones are commercially available under the trade designations LUVISKOL K12 and LUVISKOL K30 (BASF AG, Mount Olive, N.J.); and PVP K15 and PVP K30 (International Specialty Products, Inc., Wayne, N.J.). Suitable examples of polyvinylpyrrolidone/vinyl acetate copolymers are commercially available under the trade designations LUVISKOL VA28 and LUVISKOL VA73 (BASF AG); PVP/VA E-735 and PVP/VA S-630 (International Specialty Products, Inc.). Suitable examples of polyinylpyrrolidone/vinyl acetate/vinyl propionate terpolymers are commercially available under the trade designation LUVISKOL VAP343 (BASF AG). Suitable examples of polyvinylpyrrolidone/alkyl aminoacrylate copolymers are commercially available under the trade designation LUVIFLEX (BASF AG); and COPOLYMER 845, COPOLYMER 937, and COPOLYMER 958 (International Specialty Products, Inc.). Suitable examples of polyvinylpyrrolidone/acrylate/(meth)acrylic acid copolymers are commercially available under the trade designation LUVIFLEX VBM35 (BASF AG). Suitable examples of polyvinylpyrrolidone/alkyl aminoacrylate/vinyl caprolactam are commercially available under the trade designation COPOLYMER VC-713 (International Specialty Products, Inc.). A preferred hair holding polymer is polyvinylpyrrolidone/vinyl acetate copolymers where the mole ratio of vinylpyrrolidone to vinyl acetate is about 70 to about 30 moles vinylpyrrolidone to about 30 to about 70 moles vinyl acetate. More preferably the mole ratio of vinylpyrrolidone to vinyl acetate is about 70 to about 30, about 60 to about 40, about 50 to about 50, or about 30 to about 70. Most preferably, the polyvinylpyrrolidone/vinyl acetate copolymer has a mole ratio of vinylpyrrolidone to vinyl acetate of about 70 to about 30. Such polyvinylpyrrolidone/vinyl acetate copolymers are commercially available from International Specialty Products, Inc.

Other examples of hair holding polymers include acidic-vinyl-ether-based polymers, acidic-polyvinyl-acetate-based polymers, acidic acrylic polymers, basic acrylic polymers, cellulose derivatives, chitin and chitosan derivatives, and amphoteric acrylic polymers. Examples of acidic-vinyl-ether-based polymers include methyl vinyl ether/alkyl half ester of maleic anhydride copolymers, suitable examples of which are commercially available under the trade designations GANTREZ ES-225, GANTREZ ES-425, and GANTREZ SP-215 (International Specialty Products, Inc.).

Examples of acidic-polyvinyl-acetate-based polymers include vinyl acetate/crotonic acid copolymers, vinyl acetate/crotonic acid/vinyl neodecanoate copolymers, and vinyl acetate/crotonic acid/vinyl propionate copolymers. Suitable examples of vinyl acetate/crotonic acid copolymers are commercially available under the trade designations RESIN 28-1310 (National Starch Company, Bridgewater, N.J.) and LUVISET CA66 (BASF AG). Suitable examples of vinyl acetate/crotonic acid/vinyl neodecanoate copolymers are commercially available under the trade designation RESIN 28-2930 (National Starch Company). Suitable examples of vinyl acetate/crotonic acid/vinyl propionate copolymers are commercially available under the trade designation LUVISET CAP (BASF AG).

Examples of acidic acrylic polymers include (meth)acrylic acid/(meth)acrylate ester copolymers and acrylic acid/alkyl acrylate/alkylacrylamide copolymers. Suitable examples of (meth)acrylic acid/(meth)acrylate ester copolymers are commercially available under the trade designations TPLASCIZE L53P (GOO CHEMICAL Co., Ltd., Kyungki-do, Korea) and DIAHOLD (Mitsubishi Chemical Corporation, Tokyo, Korea). Suitable examples of acrylic acid/alkyl acrylate/alkylacrylamide copolymers are commercially available under the trade designations ULTRAHOLD 8 (BASF AG) and AMPHOMER V-42 (National Starch Company).

Examples of basic acrylic polymers include acrylamide-acrylate ester tetrapolymers. Suitable examples of acrylamide-acrylate ester tetrapolymers are disclosed in Japanese Patent Application (Kokai) No. HEI 2-180911. Examples of cellulose derivatives include cationic cellulose derivatives. Suitable examples of cellulose derivatives are commercially available under the trade designations CELQUAT H-100 and CELQUAT L-200 (National Starch Company). Examples of chitin and chitosan derivatives include hydroxypropylchitosan. Suitable examples of hydroxypropylchitosan are commercially available under the trade designation CHITOFILMER (Ichimaru Pharcos Co., Ltd., Gifu, Japan). Other examples of chitin and chitosan derivatives include salts of carboxymethyl chitin, carboxymethyl chitosan or chitosan, monocarboxylic acids such as pyrrolidonecarboxylic acid, lactic acid or glycolic acid or dicarboxylic acid such as adipic acid or succinic acid. Suitable examples are commercially available under the trade designations CHIMER PC (pyrrolidonecarboxylate salt) and CHITOMER L (oxalate salt) (Union Carbide Corporation, Danbury, Connecticut).

Examples of amphoteric acrylic polymers include (meth)acrylethylbetaine/alkyl (meth)acrylate copolymers and alkyl acrylate/butylaminoethyl metharylate/octylacrylamide copolymers. Examples of (meth)acrylethylbetaine/alkyl (meth)acrylate copolymers include copolymers of N-methacryloyloxyethyl N,N-dimethylammonium α-N-methylcarboxybetaine and alkyl (meth)acrylates. Suitable examples of (meth)acrylethylbetaine/alkyl (meth)acrylate copolymers are commercially available under the trade designations YUKAFORMER M-75 and YUKAFORMER SM (Mitsubishi Chemical Corporation). Examples of alkyl acrylate/butylaminoethyl metharylate/octylacrylamide copolymers include octylacrylamide/acrylatelbutylaminoethyl methacrylate copolymer. Suitable examples of alkyl acrylate/butylaminoethyl metharylate/octylacrylamide copolymers are commercially available under the trade designations AMPHOMER 28-4910 (National Starch Company).

A composition of the invention preferably includes a polyvinylpyrrolidone-based polymer, an acidic-vinyl-ether-based polymer (for instance, methyl vinyl ether/alkyl half ester of maleic anhydride copolymers), an acidic-polyvinyl-acetate-based polymer, or an amphoteric acrylic polymer (for instance, octylacrylamide/acrylate/butylaminoethyl methacrylate copolymer). Most preferably, the composition of the invention includes a polyvinylpyrrolidone-based polymer.

A composition of the invention preferably includes at least about 0.1% by weight, more preferably at least about 1.0% by weight, hair holding polymer(s), where “% by weight hair holding polymer” refers to the percent weight based on the total weight of the composition. Preferably, a composition of the invention includes no greater than about 5.0% by weight, more preferably no greater than about 7.5% by weight, most preferably no greater than about 10% by weight, hair holding polymer(s). Most preferably, a composition of the invention includes about 2.5% hair holding polymer(s).

3. Optional Components

The compositions of the invention are useful in hair care products that are used as styling aids and in hair holding products. Styling aids are used during the process of causing hair to take on a desired configuration or coiffure. Examples of styling aids include, for instance, setting lotions, setting gels, pommades, and mousses. Styling aids may be used in conjunction with mechanical devices used in styling hair, such as a mandrel. Hair holding products are typically applied to previously styled hair to freeze the hair in the desired configuration. Examples of hair holding products include hair sprays. Hair sprays may also be used as a styling aid. The compositions may take a form customarily used in the field of hair care products for topical use, such as, for example, more or less thickened or gelled liquid, cream, mousse, lotion, gel, paste, emulsion, aerosol or any other suitable form. The hair care products may include setting gels, setting lotions, mousses, sculpting lotions, hair sprays, and pommades, for example.

The compositions may contain the various traditional additives which are used in the field of the preparation of hair-care compositions for topical use, and can be chosen, for example, from UV screening agents, thickening agents, penetrating agents, antioxidants, sequestering agents, opacifying agents, buffers, surfactants chosen from nonionic surfactants such as alkyl polyglycosides, cationic surfactants, anionic surfactants and amphoteric surfactants, solubilising agents, emollients, colorants, perfumes, preservatives, or combinations thereof.

Preferably, a composition of the invention includes a diluent suitable for application to the hair. “Suitable for application to the hair” means that the diluent does not negatively affect the aesthetics of hair (e.g., shine, managability, and the like) or cause irritation to the underlying skin. One with skill in the art will recognize that the appropriate choice of diluent will depend upon the form of delivery, for example, if the hair care composition is to be left on the hair after application (as is the case with hair holding products such as hair sprays, and styling aids such as styling gels, setting gels, mousses, and pommades).

Suitable diluents for application to the hair may include a wide range of components conventionally used in hair care compositions. They can include water, an organic solvent, and a combination thereof. A suitable solvent is preferably an alcohol (including, for instance, a monohydric alcohol or a polyhydric alcohol), a hydrocarbon solvent, a halogenated hydrocarbon solvent, an ester, a silicone or silicone derivative, or a combination thereof. Preferably, an alcohol has from about 2 to about 6 carbon atoms, and can be, for instance, ethanol, isopropanol, or a combination thereof. A suitable hydrocarbon solvent can be a linear or a branched chain hydrocarbon, preferably a saturated branched chain hydrocarbon.

Preferably, a silicone or silicone deriviative includes siloxanes, such as phenyl pentamethyl disiloxane, phenylethylpentamethyl disiloxane, hexamethyl disiloxane, methoxy propylemethyl cyclotetrasiloxane, chloropropyl pentamethyl disiloxane, hydroxypropyl pentamethyl disiloxane, octamethyl cyclotetrasiloxane, decamethyl cylcopentasiloxane, or a combination thereof.

In one embodiment, when the hair care composition is a hair holding composition, or a styling aid, such as a hair spray, mousse, gel, etc., the preferred solvents include water, ethanol, a silicone-containing solvent, or a combination thereof. Mousses and aerosol/atomized hair sprays preferably include a conventional propellant to deliver the composition as a foam (mousse) or as a fine, preferably uniform, spray (aerosol/atomized hair spray). Examples of propellants include difluoroethane, dimethylether, propane, n-butane, isobutane, carbon dioxde, nitrogen, and compressed air. If included, a propellant is preferably present in a mousse in an amount of about 2% to about 30% by weight, and preferably present in an aerosol hair spray in an amount of about 15% to about 70% by weight, based on the total weight of the composition.

A hair spray composition having a low viscosity may include an emulsifier, preferably a nonionic surfactant, a cationic surfactant, an anionic surfactant, an amphoteric surfactant, or a combination thereof. In a hair spray composition, for example, an amphoteric surfactant may be used. If included, an emulsifier is preferably present in the hair care composition in an amount of about 0.01% to about 7.5% by weight of the composition.

A hair spray formulation of the invention may also contain conventional hair care adjuvants known in the art in amounts which generally range up to about 20%, preferably from about 0.01% to about 10% by weight based on the total weight of the hair care composition. Examples of adjuvants include, but are not limited to, plasticizers, silicones, emollients, emulsifiers, lubricants, wetting agents, thickness and viscosity modifiers, colorants such as dyes or tints, perfumes or fragrances, preservatives, antifoaming agents, chelating agents, polymers and resins, hair conditioners, pH adjusting agents, and the like.

A plasticizer may be added to the hair care composition of the invention. A plasticizer can modify the characteristics of the film that is formed, or can modify the film forming characteristics of a hair care composition. Examples of plasticizers include, but are not limited to, glycols, adipic esters, phthalate esters, isobutyrate esters, terephthalate esters, silicone and silicone derivatives, epoxidized butyl esters or fatty acids, epoxidized vegetable oils, glycerine as well as polymeric plasticizers. More preferred plasticizers in accordance with the invention are, for example, di-2-ethylhexyladipate or dioctyladipate (DOA), di-2-ethylhexyl phthalate or dioctyl phthalate (DOP), di-2-ethylhexyl terephthalate (DOTP), dicyclohexyl phthalate, diisononyl adipate, diisononylphthalate, n-butyl benzyl phthalate, 1,3-butylene glycol/adipic acid polyester, dialkyl adipate, dialkyl phthalate derivatives where the alkyl group is a C₁-C₁₂ alkyl group, or preferably a C₇, C₉, or C₁₂ alkyl group, di-n-hexylazelate, diphenylphthalate, tricresol phosphate, benzyl benzoate, dibutyl phosphate, tributyl phosphate, tributoxyethyl phosphate, triphenyl phosphate, butyl acetyl ricinoleate, glycerol acetyl ricinoleate, dibutyl phthalate, diethyl phthalate, dioctyl phthalate, dimethoxyethyl phthalate, diisobutyl phthalate, diamyl phthalate, dibutyl glycolate, butyl stearate, triethyl citrate, tributyl citrate, tributyl acetyl citrate, 2-hexyltriethylacetyl citrate, dibutyl tartarate, camphor, epoxidized butyl esters of linseed oil fatty acids, epoxidized linseed oil, epoxidized soya oil, propylene glycol adipate, 2,2,4-trimethyl-1,3-pentanediol diisobutyrate (TXIB), methyl abietate, cumyl acetate, dibutoxyethyl adipate, di-n-hexylazalate, glyceryl-tri-benzerate, tri-n-butylcitrate, dioctyl fumarate, triisonyl trimellitate, dioctyl isophthalate, butyl oleate, chlorinated paraffin, tricresolphosphate and dibutyl sebacate. Other plasticizers include: Dimethicone copolyol (Dow Corning 190) at 0.01-0.02%, PEG-6 capric/caprylic glyceride (SOFTIGEN 767) at 0.5-2%, DIACETIN at 1-2%, LAURAMIDE DEA (MONAMID 716) at 0.1-1%, Phenyl trimethicone (ABIL AV 20-1000) at 0.1-0.2%, propylene glycol at 1-5%, and dipropylene glycol at 1 to 5%. Examples of coalescing solvents include, but are not limited to, acetone, methyl acetate, di- or tri-propylene glycol methyl ethers.

The present invention is illustrated by the following examples. It is to be understood that the particular examples, materials, amounts, and procedures are to be interpreted broadly in accordance with the scope and spirit of the invention as set forth herein.

### EXAMPLES

All percentages, ratios, etc., in the Example are by weight/weight % unless otherwise indicated.

The ability of LARACARE A200 (GA) and the quanterized derivative (Q-GAl 88) to shield hair from moisture was evaluated. The presence of moisture disrupts the hydrogen bonds of hair and thereby causes it to become limp and lose curl and set. The shielding of hair from moisture is referred to as “improved curl retention” or “hold.” Curl retention is measured by a method similar to the method commonly referred to as the National Starch method (see, e.g., Micchelli et al., J. Soc. Cosmetic Chemists., 19, 863-880 (1968), and “Determination of the High Humidity Curl Retention of Hair Sprays,” available from National Starch Company, Bridgewater, N.J.). Results showing the presence of curl retention are indicative of increased hair holding properties.

Additional objectives of this analysis included determining the impact of GA on the curl retention of hair under humid conditions, determining the impact of GA on the curl retention performance of a commercial hair holding polymer polyvinyl pyrrolidone/vinyl acrylate (PVP/VA 735, International Specialties Products, Inc.) under humid conditions, determining the impact of Q-GA188 on the curl retention of hair under humid conditions, determining the impact of Q-GA188 on the curl retention performance of PVP/VA 735 under humid conditions, and comparing the curl retention/holding performance of Q-GA188 with that of GA.

GA (and possibly Q-GA188) were expected to impart some degree of curl retention/hold to hair solely on that basis that they are able to form a film. The ability of a material to provide curl retention under humid conditions can be thought of as being comprised of two main factors, 1.) “inherent rigidity” and 2.) “humidity resistance.” The term “inhereint rigidity” refers to the characteristic stiffness of the polymeric film. The term “humidity resistance” refers to the ability of the curl to be maintained under humid conditions. Therefore, the following scheme was employed to isolate the curl retention performance of GA or Q-GA 188 exclusively related to humidity resistance:

1. The impact of (5%) GA on curl retention was first measured vs. CONTROL(S) under relative dry conditions, where only inherent rigidity is of significance.

2. The curl retention of the same material was then measured under relatively humid conditions, where both inherent rigidity and humidity resistance are of importance.

3. Should GA or Q-GA188 have a positive effect on curl retention vs. CONTROL(S) under dry conditions, this would be attributed to its inherent rigidity.

4. Should the tresses treated with GA or Q-GA188, either alone or in combination with PVP/VA 735 show increased improvement of curl retention vs. CONTROL(S), under humid conditions, this would reflect the ability of GA to shield hair from moisture.

5. The method used to determine the impact of GA on the curl retention performance of a commercial hair holding polymer (PVP/VA 735) under humid conditions was identical to that described immediately above with the exception that the TEST was to be an aqueous solution of 2.5% PVP/VA 735 and 5% GA, while the CONTROL would vary only by the exclusion of the GA (compensated for by increased water).

Method

The curl retention of the following generalized formulations were

TABLE 1
Testing conditions

| | | NO. OF | RELATIVE |
|---|---|---|---|
| FORMULATION | | TRESSES | HUMIDITY |
| CONTROL: | WATER | 10 | LOW |
| TEST: | 5% GA | 10 | LOW |
| TEST: | 5% Q-GA188 | 10 | LOW |
| CONTROL: | WATER | 10 | HIGH |
| TEST: | 5% GA | 10 | HIGH |
| TEST: | 5% Q-GA188 | 10 | HIGH |
| CONTROL: | 2.5% PVP/VA 735 | 10 | LOW |
| TEST: | 2.5% PVP/VA 735 + 5% GA | 10 | LOW |
| TEST: | 2.5% PVP/VA 735 + | 10 | LOW |
| | 5% Q-GA188 | | |
| CONTROL: | 2.5% PVP/VA 735 | 10 | HIGH |
| TEST: | 2.5% PVP/VA 735 + 5% GA | 10 | HIGH |
| TEST: | 2.5% PVP/VA 735 + | 10 | HIGH |
| | 5% Q-GA188 | | |

| | |
|---|---|
| TOTAL: | 120 Tresses |

Preparation of Tresses

15% Grey, black hair (6″ in length), sewn and bound (with a rigid polymeric material) at the scalp end to form 7-8 inch strips, was obtained from International Hair Importers, Inc. (Bellerose, N.Y.). These strips were then cut every ½ inch to form 120 smaller tresses for use in this study. The tresses were not washed and re-used to avoid the possibility of contamination.

Preparation of Test Solutions

Test ingredients were dissolved in deionized water at room temperature with mild stirring. Lot 2A29728301 of GA (Larex, Inc., LaraCare A200), and lot CAGO90199 of Q-GA188 (Larex non-commercial preparation) were employed.

Treatment of Tresses

Each tress was shampooed twice in a 10% solution of White Rain Shampoo (Gillette, Boston, Mass.), thoroughly rinsed in deionized water and allowed to dry. Each tress was then immersed in either a Test or water Control solution for 8 seconds, with the excess Test/Control solution removed by the tress being pulled through the thumb and index finger with pressure applied. The tresses were then wound on ½ inch rollers with moderate pressure and allowed to dry.

Curl Retention Testing

Curled tresses, with rollers removed, were mounted on PLEXIGLASS boards and the length to which they have extended/drooped was measured after various intervals of exposure to environmental conditions. All tresses to be tested under the same environmental conditions, Low or High humidity, were tested concurrently—to avoid the possibility of minor changes influencing one series of Test or Control tresses differently.

High humidity environmental conditions were 90% relative humidity (RH), 23° C. Low humidity environmental conditions were 30-45% RH, 23° C. Relative humidity was measured using a Bionaire BT-254F humidity meter (Bionaire Corporation, Allendale, N.J.).

% Curl Retention was calculated by using the following equation:

$$\frac{L - {L}_{t}}{L - {L}_{o}} \times 100 = \% curl retention ,$$

where L=Length of fully extended tress, Lₜ=Length of curl after exposure at time (t), and Lₒ=Length of curl before exposure.

Results

Low Humidity Conditions

As shown in Table 2, 5% GA and 5% Q-GA188 exhibited comparable results that, in both cases, were significantly superior to those of water (i.e., “negative” control) and 2.5% PVP/VA 735 (“positive” control). Related trends were evident throughout the study becoming most evident at 72 hours.

While the addition of 5% GA or 5% Q-GA188 to 2.5% PVP/VA 735 improved the curl retention of PVP/VA 735 to that of corresponding GA-material it did not result in increase beyond that point. That is, there was no additive performance.

TABLE 2
% Curl Retention under Low Humidity Conditions, 30-45% RH, 23° C.

| Time in | | | 5% Q- | | 2.5% PVP/VA 735 | 2.5% PVP/VA 735 |
|---|---|---|---|---|---|---|
| HOURS | Water | 5% GA | GA188 | 2.5% PVP/VA 735 | and 5% GA | and 5% Q-GA188 |
| 6 | 88 | 97 | 100 | 96 | 99 | 96 |
| 24 | 72 | 94 | 96 | 90 | 96 | 95 |
| 72 | 52 | 92^{1/2} | 93^{1/2} | 84¹ | 91^{1,2} | 93^{1,2} |

¹Superior to water at 95% confidence level.
²Superior to PVP/VA 735 at 95% confidence level.

High Humidity Conditions

As shown in Table 3, analogous to the findings under Low Humidity Conditions, 5% GA and 5% Q-GA188 exhibited comparable results that, in both cases, were significantly superior to those of water (i.e., “negative” control) and 2.5% PVP/VA 735 (“positive” control). Unlike the Low Humidity results, however, the combination of 5% GA and 2.5% PVP/VA 735 resulted in curl retention that was significantly better than that of 5% GA alone, and better than that of 2.5% PVP/VA 735 alone. In other words, there was additive performance. This was not true for the combination of 5% Q-GA188 and 2.5% PVP/VA 735, which exhibited curl retention similar to that of 5% Q-GA188 alone. These results are most evident after 40 minutes of testing with differences in curl retention becoming predominantly equalized after 80 minutes.

TABLE 3
% Curl Retention under High Humidity Conditions, 90% RH, 23° C.

| Time in | | | 5% | 2.5% | 2.5% PVP/VA735 | 2.5% PVP/VA735 |
|---|---|---|---|---|---|---|
| MINUTES | Water | 5% GA | Q-GA188 | PVP/VA 735 | and 5% GA | and 5% Q-GA188 |
| 6 | 57% | 90% | 95% | 86% | 95% | 97% |
| 14 | 38 | 88 | 90 | 74 | 92 | 83 |
| 27 | 31 | 71^{1,2} | 78^{1,2} | 42¹ | 85^{1,2} | 76^{1,2} |
| 40 | 25 | 52^{1,2} | 52^{1,2} | 25 | 70^{1,2} | 48^{1,2} |
| 48 | 20 | 38^{1,2} | 33^{1,2} | 19 | 56^{1,2} | 36^{1,2} |
| 80 | 21 | 29 | 26 | 18 | 35^{1,2} | 24 |

¹Superior to Water at 95% confidence level.
²Superior to PVP/VA 735 at 95% confidence level.

Conclusions

At the concentration employed in this study both 5% GA and 5% Q-GA188 were determined to provide greater curl retention than 2.5% PVP/VA 735, both at low and high relative humidity. While this difference was more dramatic under humid conditions, it was determined to be statistically significant under both conditions.

At low humidity, the curl retention of mixtures of either GA or Q-GA188 and PVP/VA 735 was improved somewhat as compared to that of PVP/VA 735 alone, but approximately comparable to that of the GA material alone. Each of these materials improved the curl retention of PVP/VA 735 to that of corresponding GA-material only, with no increase beyond that point.

Under conditions of high humidity, however, the combination of GA and PVP/VA 735 demonstrated synergistic performance as compared with those observed at lower humidity. Unlike findings at low humidity the combination of these materials resulted in curl retention beyond that of either materials alone. This was not, however, the case of Q-GA188. Given, the ability of GA to shield various other substrates from moisture, a plausible explanation for this phenomenon is that under conditions of high humidity GA serves to shield PVP/VA 735 from the effects of moisture and thereby amplifies its performance.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition comprising an arabinogalactan and a hair holding polymer, wherein the arabinogalactan and the hair holding polymer are present in an amount effective to increase curl retention.

2. The composition of claim 1 wherein the hair holding polymer is selected from the group consisting of a polyvinylpyrrolidone-based polymer, an acidic-vinyl-ether-based polymer, an acidic-polyvinyl-acetate-based polymer, an acidic acrylic polymer, a basic acrylic polymer, a cellulose derivative, a chitin derivative, a chitosan derivative, an amphoteric acrylic polymer, and combinations thereof.

3. The composition of claim 1 wherein the composition is in the form of a hair care product selected from the group consisting of a setting lotion, a setting gel, a mousse, a hair spray, and a pommade.

4. The composition of claim 1 further comprising a plasticizer.

5. The composition of claim 1 wherein the composition comprises at least about 1% by weight arabinogalactan, based on the total weight of the composition.

6. The composition of claim 1 wherein the composition comprises at least about 0.1% by weight hair holding polymer, based on the total weight of the composition.

7. The composition of claim 1 wherein the arabinogalactan is obtained from a tree of the genus Larix.

8. A composition comprising an arabinogalactan and a polyvinylpyrrolidone-based polymer, wherein the arabinogalactan and the polymer are present in an amount effective to increase curl retention.

9. A composition comprising an arabinogalactan and a vinylpyrrolidone/vinyl acetate copolymer, wherein the arabinogalactan and the copolymer are present in an amount effective to increase curl retention.

10. A composition comprising ultrarefined arabinogalactan and a vinylpyrrolidone/vinyl acetate copolymer, wherein the arabinogalactan and the copolymer are present in an amount effective to increase curl retention.

11. A composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate, wherein the arabinogalactan and the copolymer are present in an amount effective to increase curl retention.

12. A composition comprising an ultrarefined arabinogalactan and a hair holding polymer, wherein the ultrarefined arabinogalactan and the hair holding polymer are present in an amount effective to increase curl retention.

13. A composition comprising an ultrarefined arabinogalactan and a polyvinylpyrrolidone-based polymer, wherein the ultrarefined arabinogalactan and the polymer are present in an amount effective to increase curl retention.

14. A composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate, wherein the arabinogalactan and the copolymer are present in an amount effective to increase curl retention.

15. A method for increasing curl retention, comprising:
contacting hair with an amount of a composition comprising an arabinogalactan and a hair holding polymer effective to increase curl retention; and
allowing the hair to dry.

16. The method of claim 15 wherein the hair holding polymer is selected from the group consisting of a polyvinylpyrrolidone-based polymer, an acidic-vinyl-ether-based polymer, an acidic-polyvinyl-acetate-based polymer, an acidic acrylic polymer, a basic acrylic polymer, a cellulose derivative, a chitin derivative, a chitosan derivative, an amphoteric acrylic polymer, and combinations thereof.

17. The method of claim 15 wherein the composition further comprises a plasticizer.

18. The method of claim 15 wherein the composition is in the form of a hair care product selected from the group consisting of a setting lotion, a setting gel, a mousse, a hair spray, and a pommade.

19. The method of claim 15 wherein the composition comprises at least about 1% by weight arabinogalactan, based on the total weight of the composition.

20. The method of claim 15 wherein the composition comprises at least about 0.1% by weight hair holding polymer, based on the total weight of the composition.

21. The method of claim 15 wherein the arabinogalactan is obtained from a tree of the genus Larix.

22. The method of claim 15 further comprising styling the hair.

23. The method of claim 22 wherein the hair is styled before, during, or after contacting the hair with the composition.

24. A method for increasing curl retention, comprising:
contacting hair with an amount of a composition comprising arabinogalactan and a polyvinylpyrrolidone-based polymer effective to increase curl retention; and
allowing the hair to dry.

25. A method for increasing curl retention, comprising:
contacting hair with an amount of a composition comprising arabinogalactan and a vinylpyrrolidone/vinyl acetate copolymer effective to increase curl retention; and
allowing the hair to dry.

26. A method for increasing curl retention, comprising:
contacting hair with an amount of a composition comprising ultrarefined arabinogalactan and a vinylpyrrolidone/vinyl acetate copolymer effective to increase curl retention; and
allowing the hair to dry.

27. A method for increasing curl retention, comprising:
contacting hair with a composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention; and
allowing the hair to dry.

28. A method for increasing curl retention, comprising:
contacting hair with an amount of a composition comprising an ultrarefined arabinogalactan and a hair holding polymer effective to increase curl retention; and
allowing the hair to dry.

29. A method for increasing curl retention, comprising:
contacting hair with an amount of a composition comprising an ultrarefined arabinogalactan and a polyvinylpyrrolidone-based polymer effective to increase curl retention; and
allowing the hair to dry.

30. A method for increasing curl retention, comprising:
contacting hair with a composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention; and
allowing the hair to dry.

31. A method for styling hair, comprising:
styling hair;
contacting hair with an amount of a composition comprising an arabinogalactan and a hair holding polymer effective to increase curl retention; and
allowing the hair to dry.

32. The method of claim 31 wherein the hair holding polymer is selected from the group consisting of a polyvinylpyrrolidone-based polymer, an acidic-vinyl-ether-based polymer, an acidic-polyvinyl-acetate-based polymer, an acidic acrylic polymer, a basic acrylic polymer, a cellulose derivative, a chitin derivative, a chitosan derivative, an amphoteric acrylic polymer, and combinations thereof.

33. The method of claim 31 wherein the hair is styled before, during, or after contacting the hair with the composition.

34. The method of claim 31 wherein the composition further comprises a plasticizer.

35. The composition of claim 31 wherein the composition is in the form of a hair care product selected from the group consisting of a setting lotion, a setting gel, a mousse, a hair spray, and a pommade.

36. The method of claim 31 wherein the composition comprises at least about 1% by weight arabinogalactan, based on the total weight of the composition.

37. The method of claim 31 wherein the composition comprises at least about 0.1% by weight hair holding polymer, based on the total weight of the composition.

38. The method of claim 31 wherein the arabinogalactan is obtained from a tree of the genus Larix.

39. A method for styling hair, comprising:
styling hair;
contacting the hair with an amount of a composition comprising arabinogalactan and a polyvinylpyrrolidone-based polymer effective to increase curl retention; and
allowing the hair to dry.

40. A method for styling hair, comprising:
styling hair;
contacting the hair with an amount of a composition comprising arabinogalactan and a vinylpyrrolidone/vinyl acetate copolymer effective to increase curl retention; and
allowing the hair to dry.

41. A method for styling hair, comprising:
styling hair;
contacting the hair with an amount of a composition comprising ultrarefined arabinogalactan and a vinylpyrrolidone/vinyl acetate copolymer effective to increase curl retention; and
allowing the hair to dry.

42. A method for styling hair, comprising:
styling hair;
contacting the hair with an amount of a composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention; and
allowing the hair to dry.

43. A method for styling hair, comprising:
styling hair;
contacting hair with an amount of a composition comprising an ultrarefined arabinogalactan and a hair holding polymer effective to increase curl retention; and
allowing the hair to dry.

44. A method for styling hair, comprising:
styling hair;
contacting the hair with an amount of a composition comprising an ultrarefined arabinogalactan and a polyvinylpyrrolidone-based polymer effective to increase curl retention; and
allowing the hair to dry.

45. A method for styling hair, comprising:
styling hair;
contacting the hair with an amount of a composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention; and
allowing the hair to dry.

46. A composition comprising an arabinogalactan and a hair holding polymer, wherein the arabinogalactan and the hair holding polymer are present in an amount effective to increase curl retention under high humidity.

47. A composition comprising an arabinogalactan and a polyvinylpyrrolidone-based polymer, wherein the arabinogalactan and the polymer are present in an amount effective to increase curl retention under high humidity.

48. A composition comprising an arabinogalactan and a vinylpyrrolidone/vinyl acetate copolymer, wherein the arabinogalactan and the copolymer are present in an amount effective to increase curl retention under high humidity.

49. A composition comprising ultrarefined arabinogalactan and a vinylpyrrolidone/vinyl acetate copolymer, wherein the arabinogalactan and the copolymer are present in an amount effective to increase curl retention under high humidity.

50. A composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate, wherein the arabinogalactan and the copolymer are present in an amount effective to increase curl retention under high humidity.

51. A composition comprising an ultrarefined arabinogalactan and a hair holding polymer, wherein the ultrarefined arabinogalactan and the hair holding polymer are present in an amount effective to increase curl retention.

52. A composition comprising an ultrarefined arabinogalactan and a polyvinylpyrrolidone-based polymer, wherein the ultrarefined arabinogalactan and the polymer are present in an amount effective to increase curl retention.

53. A composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate, wherein the arabinogalactan and the copolymer are present in an amount effective to increase curl retention under high humidity.

54. A method for increasing curl retention, comprising:
contacting hair with an amount of a composition comprising an arabinogalactan and a hair holding polymer effective to increase curl retention under high humidity; and
allowing the hair to dry.

55. A method for increasing curl retention, comprising:
contacting hair with an amount of a composition comprising arabinogalactan and a polyvinylpyrrolidone-based polymer effective to increase curl retention under high humidity; and
allowing the hair to dry.

56. A method for increasing curl retention, comprising:
contacting hair with an amount of a composition comprising arabinogalactan and a vinylpyrrolidone/vinyl acetate copolymer effective to increase curl retention under high humidity; and
allowing the hair to dry.

57. A method for increasing curl retention, comprising:
contacting hair with an amount of a composition comprising ultrarefined arabinogalactan and a vinylpyrrolidone/vinyl acetate copolymer effective to increase curl retention under high humidity; and
allowing the hair to dry.

58. A method for increasing curl retention, comprising:
contacting hair with an amount of a composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention under high humidity; and
allowing the hair to dry.

59. A method for increasing curl retention, comprising:
contacting hair with an amount of a composition comprising an ultrarefined arabinogalactan and a hair holding polymer effective to increase curl retention; and
allowing the hair to dry.

60. A method for increasing curl retention, comprising:
contacting hair with an amount of a composition comprising an ultrarefined arabinogalactan and a polyvinylpyrrolidone-based polymer effective to increase curl retention; and
allowing the hair to dry.

61. A method for increasing curl retention, comprising:
contacting hair with an amount of a composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention under high humidity; and
allowing the hair to dry.

62. A method for styling hair, comprising:
styling hair;
contacting hair with an amount of a composition comprising an arabinogalactan and a hair holding polymer effective to increase curl retention under high humidity; and
allowing the hair to dry.

63. A method for styling hair, comprising:
styling hair;
contacting the hair with an amount of a composition comprising arabinogalactan and a polyvinylpyrrolidone-based polymer effective to increase curl retention under high humidity; and
allowing the hair to dry.

64. A method for styling hair, comprising:
styling hair;
contacting the hair with an amount of a composition comprising arabinogalactan and a vinylpyrrolidone/vinyl acetate copolymer effective to increase curl retention under high humidity; and
allowing the hair to dry.

65. A method for styling hair, comprising:
styling hair;
contacting the hair with an amount of a composition comprising ultrarefined arabinogalactan and a vinylpyrrolidone/vinyl acetate copolymer effective to increase curl retention under high humidity; and
allowing the hair to dry.

66. A method for styling hair, comprising:
styling hair;
contacting the hair with an amount of a composition comprising arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention under high humidity; and
allowing the hair to dry.

67. A method for styling hair, comprising:
styling hair;
contacting hair with an amount of a composition comprising an ultrarefined arabinogalactan and a hair holding polymer effective to increase curl retention; and
allowing the hair to dry.

68. A method for styling hair, comprising:
styling hair;
contacting the hair with an amount of a composition comprising an ultrarefined arabinogalactan and a polyvinylpyrrolidone-based polymer effective to increase curl retention; and
allowing the hair to dry.

69. A method for styling hair, comprising:
styling hair;
contacting the hair with an amount of a composition comprising ultrarefined arabinogalactan and a vinylpyrrolidone/vinyl acetate copolymer having a vinylpyrrolidone to vinyl acetate mole ratio of about 70 to about 30 moles vinylpyrrolidone to about 30 to about 70 moles vinyl acetate effective to increase curl retention under high humidity; and
allowing the hair to dry.
